(19) United States
(12) Patent Application Publication
(10) Pub. No.: US 2002/0177580 A1
(43) Pub. Date: Nov. 28, 2002

(54) Means and method for hormonal contraception

(76) Inventors: Agatha Antonia Magdalena Van Beek, Uden (NL); Herman Jan Tijmen Coelingh Bennink, Driebergen (NL)

Correspondence Address: YOUNG & THOMPSON, 745 SOUTH 23RD STREET 2ND FLOOR, ARLINGTON, VA, 22202

(21) Appl. No.: 09/862,499

(22) Filed: May 23, 2001

(51) Int. Cl. A61K 31/56

(52) U.S. Cl. 514/171; 514/182

(57) ABSTRACT

A contraceptive method comprises the administration of a hormone composition in an amount effective to inhibit ovulation. More specifically the invention relates to the use of a hormone composition in the manufacture of a kit containing a plurality of dosage units for use in a contraceptive method, which method comprises administering a sequence of the dosage units to a female of childbearing capability so as to provide the hormone composition in an amount which is effective to inhibit ovulation, wherein the hormone composition is dydrogesterone component or a combination of estrogen and dydrogesterone component. Another embodiment concerns an oral contraceptive kit comprising from 20-35 daily oral dosage units, wherein 10-35 units contain a combination of estrogen in an amount equivalent to at least 2 μg ethinyl estradiol and dydrogesterone component in an amount equivalent to at least 2 mg dydrogesterone, 0-25 units contain estrogen in an amount equivalent to at lease 2 μg ethinyl estradiol and no progestogen, and 0-8 units contain no progestogen and no estrogen.

### TECHNICAL FIELD

The present invention is concerned with a contraceptive method, which method comprises administering to a female of childbearing capability one or more dosage units containing a hormone composition in a therapeutically effective amount to inhibit ovulation. The hormone composition used in accordance with the present invention contains a special progestogen (i.e. dydrogesterone) which is structurally and biologically closely related to progesterone and produces less undesirable side-effects than the synthetic progestogens commonly used in hormonal contraceptive regimens.

### BACKGROUND OF THE INVENTION

Dydrogesterone (9β,10α-pregna-4,6-diene-3,20-dione) is an orally active progestative hormone that has been commercially available since the early seventies. Dydrogesterone is mainly used in the treatment of endometriosis and in hormone replacement therapy.

“Martindale” (Martindale, “The Complete Drug Reference”, Micromedex Healthcare Series, Integrated Index 1974-2001) mentions a number of indications for which dydrogesterone may suitably be used. These include menstrual disorders, endometriosis, endometrial protection during menopausal hormone replacement therapy, threatened abortion, habitual abortion and infertility. Except for endometriosis all these applications have in common that they relate to progesterone deficiency in the body.

In a research publication by researchers working for the sole manufacturer of dydrogesterone (Solvay Pharma), it is reported that dydrogesterone does not inhibit ovulation in humans (Claassen V., Morsink L., de Wachter A. M., “Influence of dydrogesterone on ovulation in the rat, rabbit and monkey”, Acta Endocrinologica (1967), 67, 551-562).

U.S. Pat. No. 6,214,815 (Shangold et al.) is concerned with a triphasic oral contraceptive unit that comprises dosage units containing a combination of an estrogen and a progestogen at a contraceptively effective dosage. In a long list with examples of progestogens that may be employed in these triphasic contraceptive units, dydrogesterone is mentioned. In a table, taken from literature, recommended dosages and dosage ranges are given for a number of progestogens. The recommended dosage for dydrogesterone is reported to be 10 mg/day. The recommended dosage range is given as 5-30 mg/day.

U.S. Pat. No. 5,633,242 (Oettel) describes a method of contraception or hormone replacement beginning on the first day of menses consisting of the steps of (1) administering 3 or 4 daily doses comprising a biogenous estrogen, (2) administering 20-22 daily doses comprising a biogenous estrogen and a gestagen, and (3) administering 3 or 4 daily doses comprising a biogenous estrogen. Dydrogesterone is mentioned as an example of a gestagen that can be used in this method.

DE-A 42 24 534 (Ehrlich et al.) is concerned with a so called sequential contraceptive method that consists of one phase of 5-14 days during which an estrogen preparation is administered in an therapeutically effective amount to cause disturbance of the follicle stimulation and another phase of 14-23 days during which a combination of estrogen and progestogen preparation is administered in a therapeutically effective amount to inhibit ovulation.

### SUMMARY OF THE INVENTION

Currently on the market there are a number of contraceptive preparations which can be classified into two general types. The first are known as monophasic preparations. These contain a constant amount of estrogen and progestogen. Newer preparations known as triphasic preparations have varying levels of estrogen and progestogen; in most cases consisting of relatively constant levels of estrogen with a step-wise increase in progestogen throughout the cycle. This pattern of estrogen and progestogen administration results in a relatively dominant estrogenic formulation at the beginning of the package with increasing progestogenic activity toward the end of the package.

Almost all of the methods of hormonal contraception currently on the market have in common that they are based on a regimen which involves an administration-free interval of about 7 days whereby withdrawal bleeding, simulating the natural menses, occurs. Thus 21 day intervals of hormone administration alternate with 7 days during which no hormones are administered.

Another characteristic of these methods is the combined use of estrogen and progestogen throughout the administration regimen. So called “unopposed” administration of estrogen has been associated with endometrial proliferation in menopausal women who received estrogen replacement therapy. It is widely accepted that continuous “unopposed” estrogen therapy substantially increases the risk of endometrial cancer. In order to counteract the negative effects of unopposed estrogen therapy, adjunctive progestogen treatment is nowadays commonly applied, also in the field of hormonal contraception. Regular progestogen administration is believed to inhibit the continual estrogen stimulation of the endometrium through an anti-proliferative effect and appears to reduce the incidence of endometrial carcinoma in post-menopausal women receiving estrogen replacement therapy (Beral V., Banks E., Reeves G., Appleby P., “Use of HRT and the subsequent risk of cancer”, J. Epidemiol. Biostat. (1999) 4(3), 191-210).

Another hormonal contraceptive method, which has hardly found commercial application, is the so called sequential method. Typical of these sequential contraceptive methods is that they apply two consecutive phases, one phase which does not utilise progestogen and which is estrogen dominated and another phase which applies a combination of estrogen and progestogen during which phase the endometrium is transformed from a proliferative into a secretory state.

Yet another hormonal contraceptive method which, like the aforementioned sequential method, has found little application, is the so called “progestogen-only” method. In the progestogen-only method ovulation inhibition is achieved by administration of progestogen only, i.e. there is no co-administration of meaningful amounts of estrogen. An obvious benefit of the progestogen-only method is the fact that it does not use estrogen. As a result, side-effects associated with the use of estrogen do not occur in this method. Progestogen-only treatment is indicated for women who do not tolerate combined oral contraceptives and for those who have contraindications to the use of estrogens, e.g. women who are at increased risk of developing thrombosis. A drawback associated with progestogen-only regimens is the unpredictability of the bleeding pattern.

The three aspects that are considered to be most important in hormonal contraception are contraceptive reliability, cycle control and minimum side-effects. It is a commonly held belief that the contraceptive reliability is critically dependent on the inhibition of ovulation by the progestogen constituent. Added thereto are the peripheral effects of the progestogen on the cervix, fallopian tubes, and endometrium. In combined ethinyl estradiol/progestogen preparations the daily progestogen dose is always significantly higher compared to the ovulation inhibiting dose of the progestogen alone. This has two major reasons. Firstly the addition of ethinyl estradiol increases the level of Sex Hormone Binding Globulin (SHBG). SHBB binds and inactivates both estrogens and progestogens. The free, non SHBG bound fraction of those steroids is biologically active. Due to this mechanism a higher progestogen dose is needed in combined preparations to achieve a sufficiently high free progestogen level. Secondly when adding an estrogen to the contraceptive regimen more progestogen is needed to counteract estrogen induced endometrial proliferation (monophasic/continuous combined pills) or transform the endometrium from proliferation to secretion (sequential pills). Although estrogen itself is commonly held to be added to contraceptive regimens to achieve acceptable vaginal bleeding pattern, estrogens also inhibit ovulation in a dose dependent way. Therefore an amount of estrogen that is sufficiently biologically active, either a biogenic or a synthetic estrogen or a combination thereof, will inhibit ovulation. However biogenic estrogens alone require such high dosages for inhibition of ovulation that side-effects prevent the use of such compounds. Combined ethinyl estradiol progestogen preparations that are taken over three weeks followed by an administration pause of 6-7 days have, hitherto, shown the greatest contraceptive reliability. Thus it is not surprising that these combined preparations have gained enormous popularity.

The contraceptive method according to the invention encompasses monophasic, diphasic, triphasic, sequential as well as progestogen-only regimens and uses a specific progestogen (i.e. dydrogesterone) which until now has not found application in hormonal contraceptives. The present method also encompasses a so called “continuous combined” method, such as the one described in WO 99/12531.

As mentioned above, dydrogesterone has been commercially available since the early seventies and is successfully used in the treatment of a variety of disorders resulting from progesterone deficiency. Early research results (see the aforementioned publication by Claassen et al), which show that dydrogesterone cannot be used to inhibit ovulation as well as the product information provided by the manufacturer, have effectively discouraged researchers from using dydrogesterone in hormonal contraceptive regimens.

We have surprisingly found that, contrary to the commonly held view, dydrogesterone can successfully be used in hormonal contraceptives. In addition we have found that the use of dydrogesterone in such contraceptives offers clear advantages over the use of known synthetic progestogens such as norethindrone, levonorgestrel, norgestimate, desogestrel, drospirenone.

### DETAILED DESCRIPTION OF THE INVENTION

One embodiment of the present invention is concerned with the use of a hormone composition in the manufacture of a kit containing a plurality of dosage units for use in a contraceptive method, which method comprises administering a sequence of said dosage units to a female of childbearing capability so as to provide the hormone composition in an amount which is effective to inhibit ovulation, wherein the hormone composition is dydrogesterone component or a combination of estrogen and dydrogesterone component.

Throughout is document the term “dydrogesterone component” encompasses dydrogesterone, dydrogesterone precursors, dydrogesterone metabolites, and mixtures thereof. By precursors of active ingredients, such as dydrogesterone, are meant components capable of liberating the active ingredient when used in the present contraceptive method, particularly after administration, e.g. as a result of metabolic conversion of the precursor substance. The term “dydrogesterone metabolites” relates to substances that are produced with the human body, as a result of metabolic activity, after administration of a dydrogesterone component and which display a hormonal functionality which is comparable to that of the dydrogesterone component. In this context is noted that a widely used progestogen, levonorgestrel, is a metabolite of another popular progestogen, norgestimate.

Dydrogesterone (9β,10α-pregna-4,6-diene-3,20-dione) is structurally more closely related to progesterone (Pregn-4-ene-3,20-dione) than any other synthetic progestogen that is currently used in commercially available hormonal contraceptives. Dydrogesterone is a structural isomer of progesterone that contains an additional unsaturation. Unlike the synthetic progestogens that are used commercially, dydrogesterone does not exhibit estrogenic, glucocorticoid, anabolic or androgenic properties and thus provides a metabolic profile which is more favourable. Thus, in comparison to existing progestogen-containing contraceptive kits, the kit according to the present invention offers the advantage of less undesired side-effects especially on lipid and carbohydrate metabolism (Van der Mooren et al., “A 2 year study on the beneficial effects of 17beta-oestradiol—dydrogesterone therapy in postmenopausal women”, Eur. J. Gynecol. Reprod. Biol. (1993) 52(2), 117-123 and Crook et al., “Hormone replacement therapy with dydrogesterone and 17beta-oestradiol: effects on serum lipoproteins and glucose tolerance during 24 month follow up”, Br J Obstet Gynaecol (1997) 104(3), 298-304). Moreover there are no adverse effects on blood pressure (Grisar et al., “Hormone replacement therapy with 17beta-estradiol dydrogesterone”, Wien Klin. Wochenschr. (1999), 111(24), 1035-1043).

As mentioned herein before the present kit can suitably be used in monophasic, diphasic, triphasic, sequential as well as in progestogen-only contraceptive methods. All these regimens have in common that during at least part of the regimen a progestogen is applied. The kit according to the invention may also suitably be utilised in contraceptive methods that are not based on a 4 week menstruation cycle, such as the “continuous combined” method described in WO 99/12531 (Hesch).

In a preferred embodiment of the invention the dosage units contained in the present kit are daily dosage units. The present method preferably comprises administering a sequence of at least 10 such daily dosage units containing the combination of estrogen and dydrogesterone component.

In a preferred embodiment of the invention the hormone composition used in the contraceptive method is a combination of estrogen and dydrogesterone component. This embodiment includes monophasic, diphasic, triphasic, continuous combined as well as sequential methods.

Examples of estrogens (synthetic and biogenic) which may suitably be used in the present invention include ethinyl estradiol, mestranol, quinestranol, estradiol, estrone, estran, estriol, estetrol, conjugated equine estrogens, precursors capable of liberating such an estrogen when used in the present method and mixtures thereof

It was found that usually the equivalent of a daily oral dosage of at least 2 mg dydrogesterone is required to obtain good contraceptive reliability and cycle control. Hence, in a preferred embodiment, the dosage units contain the dydrogesterone component in an amount which is equivalent to at least 2 mg dydrogesterone. More preferably the dosage units contain the dydrogesterone component in an amount equivalent to 3-50 mg dydrogesterone. Even more preferred is the use of the dydrogesterone component in a daily dosage which is equivalent to 5-30 mg dydrogesterone. The optimum balance between contraceptive reliability, cycle control and side-effects is obtained when the units comprise the dydrogesterone component in an amount equivalent to 10-20 mg dydrogesterone.

In accordance with the invention the amount of estrogen contained in the dosage units usually exceeds the equivalent of 2 μg ethinyl estradiol. Preferably the dosage units contain estrogen in an amount equivalent to 4-40 μg ethinyl estradiol.

The term female, whenever referred to in here, relates to female mammals. Preferably the female mammal is a homo sapiens. For homo sapiens females are usually biologically capable of child bearing between the age of 12 and 55.

The dosage units according to the invention may be administered orally, parenterally, sublingually, transdermally, intravaginally, intranasally or buccally. The daily hormonal units can suitably be administered orally, transdermally or intravaginally. Methods for transdermal administration including the associated method for manufacturing such systems are well known in the art. In this connection, reference may be had to U.S. Pat. Nos. 4,752,478, 4,685,911, 4,438,139 and 4,291,014. Most preferably the dosage units according to the invention are oral dosage units.

For most human females the natural interval between menses is somewhere between 20 and 35 days. To mimic the natural cyclic menses pattern, it is preferred that the plurality of daily dosage units consists of 20 to 35 dosage units. Most preferably the plurality of daily dosage units consists of 28 dosage units.

In a preferred embodiment the present kit is used in a sequential contraceptive method which comprises at least two phases, an estrogenic and a progestogenic phase, said method comprising administering

a) during the estrogenic phase one or more dosage units to provide a therapeutically effective amount of estrogen to inhibit ovulation,

b) during the progestogenic phase one or more dosage units to provide a combination of dydrogesterone component and estrogen in a therapeutically effective amount to inhibit ovulation and to transform the endometrium from a proliferative into a secretory state, and optionally

c) during a hormone-free phase one or more placebo's.

It is noted that if the regimen includes a hormone-free phase, said phase will follow the progestogenic phase as otherwise the bleeding pattern may be adversely affected. The aforementioned sequential method preferably involves no major administration-free intervals. This sequential method allows administration-free intervals of up to 2 days without a serious decrease in reliability. Hence the method encompasses a regimen which includes one or more administration-free intervals of up to 2 days. In a more preferred embodiment, the contraceptive method comprises the uninterrupted daily administration of a hormone unit from the plurality of daily hormone units during the cycle between 2 menses, more preferably during at least 3 of such cycles.

In terms of contraceptive reliability the sequential method of the invention without administration-free intervals performs better than methods that make use of combined preparations. Due to the fact that the present sequential method does not make use of administration-free intervals, the risk that mistakes in the administration will lead to escape ovulations is much lower than in methods using combined preparations and an administration-free interval. The combination of a pause of 6-7 days during which significant follicular development occurs and the well documented bad compliance of many pill-users (30%-40% forget pills occasionally) cause an increased risk of escape ovulation “around” the pill pause. This results in “real life” pregnancy rates of 3-8% per year. By removing the pause and administering ovulation inhibiting steroids daily, the risk of escape ovulation is much lower with the proposed newly invented regimen.

In the above sequential method both biogenic and synthetic estrogens may be used. Thus, during the estrogenic phase, the dosage units may comprise synthetic estrogen, biogenic estrogen or a combination of synthetic and biogenic estrogen in an effective amount to inhibit ovulation. According to a preferred embodiment the estrogen contained in the units for use in the estrogenic phase is selected from the group consisting of synthetic estrogen and a mixture of synthetic estrogen and biogenic estrogen as the use of a synthetic estrogen provides highest contraceptive reliability. It was found to be particularly advantageous to administer a combination of synthetic and biogenic estrogen as this enables a reduction of the dose of synthetic estrogen needed to achieve ovulation inhibition whilst at the same time maintaining maximum contraceptive reliability. With synthetic estrogens there is a (dose dependent) risk of undesirable side-effects. Examples of side effects associated with the administration of estrogens are nausea, vomiting, breast tension, headache, mood disturbances, fluid retention, bloating, liver function disturbances, cholelithiasis, cholestatic icterus, pancreatitis and thromboembolism.

In addition, during the estrogenic phase, the dosage units preferably do not contain progestogen as the presence of such hormone may adversely affect the bleeding pattern. Most preferably the units containing the synthetic estrogen are free of progestogen, anti-progestogen and androgen.

In the present method, during the progestogenic phase, dosage units are administered to provide a combination of estrogen and dydrogesterone component in a therapeutically effective amount to inhibit ovulation and to transform the endometrium from a proliferative into a secretory state. In a particularly preferred embodiment of the invention the units containing a combination of estrogen and progestogen do not contain a synthetic estrogen, i.e. the estrogen is biogenic estrogen. During the progestogenic phase adequate reliability can be achieved without the use of a synthetic estrogen like ethinyl estradiol. Because biogenic estrogens are naturally present in the female body, side-effects do not normally occur as long as serum levels do not substantially exceed naturally occurring concentrations. In the present method also side-effects that occur as a result of chronic fluctuations in blood serum estrogen levels, e.g. estrogen withdrawal symptoms, are avoided.

In the aforementioned embodiment of the invention a combination of biogenic estrogen and dydrogesterone component is applied during the progestogenic phase. In such an embodiment not only does the hormonal pattern observed during the menstruation cycle closely resemble that found in natural female physiology, but in addition the combination of biogenic estrogen and dydrogesterone component (the synthetic progestogen most closely resembling biogenic progesterone) is very similar to the combination of hormones which naturally dominate the secretory phase of the natural cycle.

The administration of estrogen as the only hormonally active ingredient during the estrogenic phase does not interfere with the withdrawal bleeding which occurs after the progestogenic phase due to discontinuation (withdrawal) of the progestogen administration. Unopposed estrogen administration causes stimulation of the progesterone receptors in the endometrium, allowing progestogens to be optimally effective in transforming the endometrium in a successive phase. As a result of this, a reduced rate of intermenstrual breakthrough bleeding, compared to conventionally combined low-dose preparations, is achieved. It is preferred to employ synthetic, or a combination of synthetic and biogenic estrogen during the estrogenic phase so as to ensure an optimum level of contraceptive reliability.

In a preferred embodiment of the invention the daily dosage units for use during the estrogenic phase contain the synthetic estrogen in an amount equivalent to 3-40 μg ethinyl estradiol. It is an important aspect of the present invention that it enables the use of synthetic estrogen at very reduced levels without a significant reduction in contraceptive reliability or a deterioration of bleeding pattern. Whereas most commercially available kits comprise daily dosage units that contain more than 15 μg ethinyl estradiol, the present invention achieves contraceptive reliability at lower amounts. Hence, preferably, the daily dosage units for use during the estrogenic phase contain the synthetic estrogen in an amount equivalent to 5-15 μg ethinyl estradiol.

The daily dosage units for use during the progestogenic phase preferably contain the biogenic estrogen in an amount equivalent to 0.5-5 mg 17beta-estradiol. The invention makes it possible to apply relatively low levels of biogenic estrogen. Thus in another preferred embodiment the daily dosage units for use during the progestogenic phase contain the biogenic estrogen in an amount equivalent to 1-3 mg 17beta-estradiol.

The daily dosage units for use during the progestogenic phase may suitably contain the dydrogesterone component in an amount equivalent to 3-50 mg dydrogesterone.

Another preferred embodiment of the invention relates to a continuous combined contraceptive method, which method comprises the continuous administration of dosage units containing a combination of estrogen and dydrogesterone component during a period of at least 6 weeks, preferably at least 9 weeks, so as to provide a combination of estrogen and dydrogesterone component in an amount effective to inhibit ovulation during said period. An example of a continuous combined contraceptive method may be found in WO 99/12531 (Hesch). In the present continuous combined method the estrogen preferably consists of synthetic estrogen or a combination of synthetic and biogenic estrogen. The continuous combined method offers the advantage that it enables the application of very low dosages of estrogen. Hence in a preferred embodiment of the continuous combined method, the dosage units are daily dosage units containing estrogen in an amount equivalent to 5-15 μg ethinyl estradiol. Preferably the daily dosage units in the continuous combined method contain dydrogesterone component in an amount equivalent to 10-20 mg dydrogesterone.

Another embodiment of the invention concerns a progestogen-only method, wherein the hormone composition provided by the sequence of dosage units is dydrogesterone component and wherein the dosage units contain the dydrogesterone component in an amount equivalent to at least 2 mg dydrogesterone, preferably 3-50 mg dydrogesterone. Most preferably the dosage units used in the progestogen-only method contain the dydrogesterone component in an amount equivalent to 5-30 mg dydrogesterone.

In order to determine for a specific biogenic estrogen or synthetic estrogen the amounts that are equivalent to a cited amount of ethinyl estradiol or 17beta-estradiol, the method described by Allen and Doisy may suitably be used (Allen A., Doisy E. A., “An ovarian hormone. Preliminary report on its localization, extraction and partial purification, and action in test animals. JAMA (1923), 81, 819-821). Simiarly, in order to determine for a given progestogen the amount equivalent to cited amount of levonorgestrel the method originally described by McPhail can be used (Mc Phail M. K. “The assay of progestin” J Physiol (1934), 83, 145-156). A more recent description of this method can be found in an article written by Overbeek G. A., de Visser J. “A new substance with progestational activity”, Acta Endocrinol (1956), 22, 318-329. It is noted that the aforementioned methods will provide useful indications about the anticipated estrogenic potency or progestogenic potency of a particular hormone. However, to accurately determine the equivalent amounts that are referred to above, it is advisable to additionally conduct in vivo studies in human females.

The synthetic estrogen present in the kit according to the invention is preferably selected from the group consisting of: ethinyl estradiol, mestranol, quinestranol, precursors capable of liberating such an estrogen when used in the present contraceptive method and mixtures thereof. Most preferably the synthetic estrogen in the present kit is ethinyl estradiol or a precursor thereof. The biogenic estrogen is preferably selected from the group consisting of: estradiol, estrone, estran, estriol, estetrol, conjugated equine estrogens, precursors capable of liberating such an estrogen when used in the present method and mixtures thereof. The preferred biogenic estrogen is estradiol or a precursor thereof. Here the term estradiol encompasses both 17 alpha-estradiol and 17beta-estradiol. Most preferably the biogenic estrogen is 17beta-estradiol or a precursor thereof.

Best results are obtained with the kit according to the invention when the synthetic estrogen is ethinyl estradiol or a precursor thereof, the biogenic estrogen is estradiol or a precursor thereof. Thus in a preferred embodiment the dosage units for use during the estrogenic phase contain ethinyl estradiol or a combination of ethinyl estradiol and estradiol and/or precursors thereof in a therapeutically effective amount to inhibit ovulation and the dosage units for use during the progestogenic phase contain a therapeutically effective amount of a combination of estradiol and/or a precursor thereof and dydrogesterone component, to inhibit ovulation and to transform the endometrium from a proliferative into a secretory state.

Particularly useful precursors of the hormones present in the kit according to the invention are substances that differ from these hormones in that the hydrogen in at least one of the hydroxyl groups in the hormone-molecule has been substituted by —CO—R, wherein R is a hydrocarbon radical comprising from 1-25 carbons.

It is to be understood that the present invention not only encompasses the use of estrogens specifically mentioned in this application, but also metabolites of these hormones that display comparable functionality. In this context it is noted that estriol is a metabolite of 17beta-estradiol. Both these estrogens have found application in preparations for hormone replacement therapy.

The plurality of dosage units contained by the present kit for use in the sequential method can suitably consist of 1-18 units for use in the estrogenic phase and 10-27 units for use in the progestogenic phase and 0-10 units for use in the hormone-free phase. In the sequential method according to the invention a hormone-free phase of up to 10 days may be utilised. It is preferred, however, to minimise the duration of the hormone-free phase to less than 4 days. Thus the plurality of dosage units preferably contains no more than 4 units for use in the hormone-free phase. More preferably they contain at most 1 unit, and most preferably not a single unit for use in the hormone-free phase.

Another embodiment of the invention relates to an oral contraceptive kit comprising from 20-35 daily oral dosage units, wherein 10-35 units contain a combination of estrogen in an amount equivalent to at least 2 μg ethinyl estradiol and dydrogesterone component in an amount equivalent to at least 2 mg dydrogesterone, 0-25 units contain estrogen in an amount equivalent to at least 2 μethinyl estradiol and no progestogen, and 0-8 units contain no progestogen and no estrogen.

Preferably the oral contraceptive kit comprises 10-34 units that contain a combination of estrogen and dydrogesterone component and 1-18 units that contain estrogen and no progestogen. More preferably the kit contains 10-20 units contain a combination of estrogen and dydrogesterone component, 10-18 units containing estrogen and no progestogen and at most 1 unit containing no progestogen and no estrogen. Most preferably the kit contains 13-15 units containing a combination of estrogen and dydrogesterone component and 13-15 units containing estrogen and no progestogen.

The dosage units are preferably for oral administration and arranged in a fixed sequence corresponding to the intended order of administration in 2 phases. Preferably, the dosage units to be used in either the estrogenic or progestogenic phase are easily distinguishable, e.g. because they are different in colour and/or shape. Data indications may be provided on the packaging. The packaging may be a tube or box or a strip. The box may be circular, square, or otherwise shaped with the tablets being accommodated separately therein for ease of administration. Date indications may appear adjacent to each tablet corresponding with the days on which each tablet is to be taken. Some indication of the sequence in which the tablets are to be taken preferably appears on the packaging regardless of its form.

Generally speaking, the dosage units in the present kit are prepared according to conventionally known procedures in accordance with the method of administration. Thus, the active ingredients are prepared according to known methods in a pharmaceutically acceptable form for administration. These ingredients, in their required quantities are combined with the appropriate pharmaceutical carriers such as additives, vehicles and/or flavour ameliorating substances. These substances may be referred to as diluents, binders and lubricants. Gums, starches and sugars are also common terms. Typical of these types of substances or excipients are pharmaceutical grades of mannitol, lactose starch, magnesium stearate, sodium saccharin, talcum, cellulose, glucose, sucrose, magnesium carbonate and the like. The active ingredient(s) may comprise from about 0.01% by weight to about 99.99% by weight of the total formulation and the remainder comprises the pharmaceutically acceptable carrier. The percentage of active ingredient(s) may vary according to the delivery system or method of administration and is chosen in accordance with conventional methods known in the art. Thus, the active ingredients are compounded with the chosen carrier and in for example the case of a tablet form, placed in a tablet moulding apparatus to form the tablets which are subsequently packaged in accordance with the chosen regimen.

In the following examples, specific embodiments of the present invention are set forth. These are meat to be illustrative of the invention and are not meant to limit it in any way.

### EXAMPLES

### Example 1

Women participating in the study described in these examples are all selected on the basis that they are users of ‘high’ dose monophasic ethinyl estradiol-containing combined oral contraceptives. The reasons for applying this selection criterion is that there is a dose-relationship between follicular development and ethinyl estradiol whether given alone or in combination with a progestogen, i.e. less follicular development in the presence of the higher dose. Participation of the females involved in the study starts at a moment of (almost) complete suppression of follicular development. This condition is not met if a female has her first day of bleeding in a natural cycle, after a tablet-free interval, or immediately after a low-dose combined oral contraceptive.

A clinical study in contraception is conducted in 16 young healthy women who previously used a monophasic oral contraceptive pill with at least 30 microgram of ethinyl estradiol. Eight women are administered ethinyl estradiol at a dose of 10 microgram/day during days 1-14 of the cycle. Another eight women are administered 15 microgram/day of ethinyl estradiol during days 1-14 of the cycle. From days 15-28 of the cycle, 10 mg of dydrogesterone combined with 1 mg 17beta-estradiol is administered to the women of both (ethinyl estradiol) dose groups. The participants are followed by vaginal ultrasonography and blood sampling for endogenous hormones to assess ovarian function (ovulation inhibition). Vaginal bleeding pattern and feeling of well being are scored by the participants.

Results show that the ovarian function is suppressed to the extent of ovulation inhibition. The follicular development is more profoundly suppressed in the 15 microgram ethinyl estradiol group than in the 10 microgram group. There is no intermenstrual bleeding in either group and participants reported an improved feeling of well being.

### Example 2

A clinical study is conducted in 8 young healthy women who were selected on the basis of the same criteria as mentioned in Example 1.

The women are administered 2 mg 17beta-estradiol during days 1-14 of the cycle. From days 15 to 28 of the cycle 10 mg dydrogesterone combined with 2 mg 17beta-estradiol is administered. The participants are followed by vaginal ultrasonography and blood sampling for endogenous hormones to assess ovarian function (ovulation inhibition). Vaginal bleeding pattern and feeling of well being are scored by the participants. No intermenstual bleeding is observed and an improved feeling of well being is reported by the women.

### Example 3

A clinical study is conducted in 16 young healthy women who were selected on the basis of the same criteria as mentioned in Example 1. To 8 women a combination of 5 μg ethinyl estradiol and 10 mg dydrogesterone is administered uninterruptedly on a daily basis during a period of 4 months. The other 8 women received on a daily basis a combination of 10 μg ethinyl estradiol and 20 mg dydrogesterone, also uninterruptedly during a period of 4 months.

In both groups of women ovulation inhibition is observed during the period of the study. In the higher dose group thee is indication of more profound ovarian suppression and less breakthrough bleeding.

### Example 4

A clinical study in contraception is conducted in 8 women, who were selected on the basis of the criteria mentioned in Example 1, using 10 μethinyl estradiol and 20 mg dydrogesterone. The aforementioned dose is administered on a daily basis during 24 days, followed by a hormone-free period (placebo's) of 4 days. Regular withdrawal bleeding occurs in all women and ovulation inhibition is achieved.

### Example 5

A clinical study with dydrogesterone as a progestogen-only preparation is conducted in 8 women who were selected on the basis of the criteria mentioned in Example 1. The 8 women receive a 20 mg dosage of dydrogesterone once a day, uninterruptedly during a period of 4 months. Ovulation is inhibited and there is a reduction of the usual menses flow, despite some unpredictable irregular bleeding.

1. A contraceptive method which uses a plurality of dosage units containing a hormone composition, which method comprises administering a sequence of said dosage units to a female of childbearing capability so as to provide the hormone composition in an amount which is effective to inhibit ovulation, wherein the hormone composition is dydrogesterone component or a combination of estrogen and dydrogesterone component.

2. Method according to claim 1, wherein the hormone composition is a combination of estrogen and dydrogesterone component.

3. Method according to claim 1, where the dosage units are daily dosage units.

4. Method according to claim 3, wherein the method comprises administering a sequence of at least 10 daily dosage units containing the combination of estrogen and dydrogesterone component.

5. Method according to claim 1, wherein the dosage units contain the dydrogesterone component in an amount equivalent to at least 2 mg dydrogesterone

6. Method according to claim 5, wherein the dosage units containing the dydrogesterone component in an amount equivalent to 3-50 mg, preferably 5-30 mg dydrogesterone.

7. Method according to claim 1, wherein the dosage units containing the combination of estrogen and dydrogesterone component comprise estrogen in amount which is equivalent to at least 2 μg of ethinyl estradiol.

8. Method according to claim 1, wherein the method comprises at least two phases, an estrogen and a progestogenic phase, said method comprising administering
a) during the estrogenic phase one or more dosage units to provide a therapeutically effective amount of estrogen to inhibit ovulation,
b) during the progestogenic phase one or more dosage units to provide a combination of dydrogesterone component and estrogen in a therapeutically effective amount to inhibit ovulation and to transform the endometrium from a proliferative into a secretory state, and optionally
c) during a hormone-free phase one or more placebo's.

9. Method according to claim 8, wherein the estrogen contained in the units for use in the estrogenic phase is selected from the group consisting of synthetic estrogen or a mixture of synthetic estrogen and biogenic estrogen.

10. Method according to claim 8 or 9, wherein the daily dosage units for use during the estrogenic phase contain synthetic estrogen in an amount equivalent to 3-40 μg, preferably 5-15 μg ethinyl estradiol.

11. Method according to any one of claims 8-10, wherein the daily dosage units for use during the progestogenic phase contain biogenic estogen in an amount equivalent to 0.5-5 mg, preferably 1-3 mg 17beta-estradiol.

12. Method according to any one of claims 8-11, wherein the plurality of daily dosage units consists of 1-18 units for use in the estrogenic phase and 10-27 units for use in the progestogenic phase and 0-10 units for use in the hormone-free phase.

13. Method according to any one of claims 1-7, wherein the method comprises the continuous administration of dosage units containing a combination of estrogen and dydrogesterone component during a period of at least 6 weeks, preferably at least 9 weeks, so as to provide a combination of estrogen and dydrogesterone component in an amount effective to inhibit ovulation during said period.

14. Method according to any one of claims 1-13, wherein the estrogen is selected from the group consisting of: ethinyl estradiol, mestranol, quinestranol, estradiol, estrone, estran, estriol, estetrol, conjugated equine estrogens, precursors capable of liberating such an estrogen when used in the present method and mixtures thereof.

15. Method according to claim any one of claims 3-14, wherein the plurality of daily dosage units consists of 20 to 35 dosage units, preferably 28 dosage units.

16. Method according to claim, 1, wherein the hormone composition is dydrogesterone component and wherein the dosage units contain the dydrogesterone component in an amount equivalent to at least 2 mg dydrogesterone, preferably 3-50 mg dydrogesterone.

17. An oral contraceptive kit comprising from 20-35 daily oral dosage units, wherein 10-35 units contain a combination of estrogen in an amount equivalent to at least 2 μg ethinyl estradiol and dydrogesterone component in an amount equivalent to at least 2 mg dydrogesterone, 0-25 units contain estrogen in an amount equivalent to at least 2 μg ethinyl estradiol and no progestogen, and 0-8 units contain no progestogen and no estrogen.

18. An oral contraceptive kit according to claim 17, wherein 10-34 units contain a combination of estrogen and dydrogesterone component and 1-18 units contain estrogen and no progestogen.

19. An oral contraceptive kit according to claim 18, wherein 10-20 units contain a combination of estrogen and dydrogesterone component, 10-18 units contain estrogen and no progestogen and at most 1 unit contains no progestogen and no estrogen.
